(19) United States
(12) Patent Application Publication
(10) Pub. No.: US 2026/0115350 A1
(43) Pub. Date: Apr. 30, 2026

(54) ANTIMICROBIAL CYANOACRYLATE FORMULATIONS

(71) Applicant: Chemence Medical, Inc., Alpharetta, GA (US)

(72) Inventors: Stephen Hynes, Roswell, GA (US); David Kim, Marietta, GA (US); Sundari Rallapalli, Johns Creek, GA (US)

(73) Assignee: Chemence Medical, Inc., Alpharetta, GA (US)

(21) Appl. No.: 19/124,286

(22) PCT Filed: Oct. 25, 2023
(86) PCT No.: PCT/US2023/077792

§ 371 Date: Apr. 24, 2025

Related U.S. Application Data
(60) Provisional application No. 63/380,905, filed on Oct. 25, 2022.

(51) Int. Cl. A61L 24/00; A61L 24/06

(52) U.S. Cl. CPC A61L 24/0015; A61L 24/0021; A61L 24/06

(57) ABSTRACT

Described herein are polymerizable compositions useful as surgical adhesives, where the compositions include at least one antimicrobial agent dissolved in a cyanoacrylate monomer, where the at least one antimicrobial agent includes a cinnamon derivative comprising at least one of cinnamaldehyde, a cinnamic ester, a cinnamon essential oil, and where the at least one antimicrobial agent optionally further includes triclosan or a derivative thereof and/or povidone iodine. The antimicrobial agent is soluble in and compatible with the cyanoacrylate monomer.

### CROSS-REFERENCES TO RELATED APPLICATIONS

This application claims the benefit of U.S. Provisional Patent Application No. 63/380,905, filed Oct. 25, 2022, which is incorporated by reference herein in its entirety.

### FIELD

The present disclosure relates to surgical adhesive formulations including antimicrobial agents dissolved in cyanoacrylate monomers.

### BACKGROUND

During recovery from a surgical procedure, the most common problem a patient encounters is an infection at the surgical site. Cyanoacrylate skin adhesives are used to close wounds and prevent infection. Cyanoacrylate monomer is applied to an approximated incision or other wound as a liquid, but the monomer quickly polymerizes to form a film that acts as a barrier to external microorganisms. These cyanoacrylate adhesives are beneficial for patient care and recovery due to the bacteriostatic nature of the polymer formed and the barrier properties of the film. In many cases, however, the antimicrobial activity inherent in the cyanoacrylate monomer or polymer is insufficient, especially against G(−) strains, and an additional antimicrobial agent would be useful. Unfortunately, however, many antimicrobial agents have chemical structures that react with cyanoacrylate and/or the antimicrobial agents have very low solubility in cyanoacrylate monomers. Accordingly, combining antimicrobial agents with cyanoacrylate monomers has been challenging.

There is a need for formulations that include a polymerizable cyanoacrylate monomer and an antimicrobial having a broad spectrum of activity, where the antimicrobial is soluble in the cyanoacrylate monomer and compatible with the cyanoacrylate monomer, so the antimicrobial does not adversely affect the cyanoacrylate monomer or its polymerization.

### SUMMARY

The present disclosure provides topical adhesive solutions comprising at least one antimicrobial compound dissolved in a cyanoacrylate monomer. The at least one antimicrobial compound comprises a cinnamon derivative selected from cinnamaldehyde, a cinnamic ester, or a cinnamon essential oil. Optionally, the cinnamon derivative is present in a range from about 3% to about 35% by weight, such as from about 3% to about 10% by weight. In some embodiments, the cinnamon derivative comprises cinnamaldehyde or a cinnamon essential oil. Optionally, the cinnamaldehyde or cinnamon essential oil is present in a range from about 2% to about 30% by weight, such as from about 3% to about 10% by weight.

In some embodiments, in addition to the cinnamon derivative, for example in addition to the cinnamaldehyde or cinnamon essential oil, the at least one antimicrobial compound further comprises triclosan or a derivative thereof. In any embodiment where triclosan or derivative thereof is present, the triclosan or derivative optionally is present in a range from about 100 ppm to about 10,000 ppm, such as from about 1000 ppm to about 5000 ppm.

In some embodiments, in addition to the cinnamon derivative, and with or without the triclosan or derivative thereof, the at least one antimicrobial compound further comprises povidone iodine. In any embodiment where the povidone iodine is present, the povidone iodine optionally is present in a range from about 0.5% to about 3% by weight, such as from about 0.5% to about 2% by weight. Optionally, the topical adhesive solution further includes polyethylene glycol. When present, the polyethylene glycol optionally is present in a range from about 1% to about 10% by weight, such as from about 1% to about 5% by weight.

Any formulation disclosed herein optionally can include one or more further elements, such as but not limited to a thickener, a polymerization accelerator, a plasticizer, or a polymerization inhibitor. Optionally, any formulation disclosed herein can be devoid of a plasticizer.

When a thickener is present, optionally, the thickener comprises poly(2-ethylhexyl methacrylate), poly(2-ethylhexyl acrylate), or poly(2-octyl cyanoacrylate). When a polymerization accelerator is present, optionally, the polymerization accelerator comprises a crown ether or a calixarene. When a plasticizer is present, optionally the plasticizer comprises dibutyl sebacate, tributyl citrate, or tributyl O-acetyl citrate. When a polymerization inhibitor is present, optionally the polymerization inhibitor comprises butylated hydroxy anisole or sulfur dioxide.

In some embodiments, the formulations disclosed herein are homogenous. In some embodiments, the formulations disclosed herein are stable.

### BRIEF DESCRIPTION OF THE DRAWINGS

FIG. 1 is a graph of viscosity and cure speed at different elapsed times for an example formulation and a control formulation, where data is provided for real-time aging (at room temperature) and for accelerated aging (at 60° C.).

FIG. 2 is a graph comparing viscosities of a control formulation and two example formulations at a time pre-sterilization and at three times post-sterilization.

FIG. 3 is a graph comparing cure time for a control formulation and two example formulations according to some examples described herein.

FIG. 4 is a graph of cinnamaldehyde released (μg/g) from a cured film formed from an example formulation during a 14-day degeneration study.

### DETAILED DESCRIPTION

Provided herein are adhesive formulations for application to skin, for use in closing and securing wounds, such as surgical incisions. The formulations are solutions of antimicrobial agents dissolved in cyanoacrylate monomer. The solutions are stable and homogenous, and the dissolved antimicrobial agents do not adversely affect the cyanoacrylate monomer, do not cause premature polymerization of the cyanoacrylate, and do not prevent polymerization of the monomer after it is applied to skin. The antimicrobial agents provide broad-spectrum activity, which enhances the inherent bacteriostatic nature of the cyanoacrylate monomer and polymer. In some examples, the formulations achieve a synergistic antimicrobial effect of two or more antimicrobial agents.

Formulations are provided herein that include a cinnamon derivative, such as cinnamaldehyde, a cinnamic ester, a cinnamon essential oil, or a combination thereof, dissolved in cyanoacrylate monomer. The formulations are homogeneous, stable, and exhibit increased antimicrobial activity as compared to formulations without the cinnamon derivative.

In some embodiments, a formulation is provided that includes both a cinnamon derivative and triclosan or a derivative thereof (including antimicrobial phenol derivatives other than triclosan), dissolved in cyanoacrylate monomer. The formulation achieves a synergistic antimicrobial effect in that the antimicrobial activity of the combination is enhanced relative to the combined contribution of the individual antimicrobial agents.

In some embodiments, any formulation described herein optionally includes povidone iodine (PVP-I₂) dissolved in the cyanoacrylate monomer. When present, the PVP-I₂ is fully dissolved in the cyanoacrylate and the solution is stable. When the formulation includes PVP-I₂ the formulation can also include polyethylene glycol (PEG).

The cyanoacrylate monomer in the inventive formulations is not particularly limited and can be any alpha-cyanoacrylate monomer. As non-limiting examples, the cyanoacrylate monomer can be n-octyl cyanoacrylate, n-hexyl cyanoacrylate, adamantyl cyanoacrylate, ethyl cyanoacrylate, 2-octyl cyanoacrylate, dodecyl cyanoacrylate, 2-ethylhexyl cyanoacrylate, butyl cyanoacrylate, methyl cyanoacrylate, 3-methoxy butyl cyanoacrylate, 2-butoxyethyl cyanoacrylate, 2-isopropoxyethyl cyanoacrylate, or 1-methoxy-2-propyl cyanoacrylate. In some examples, the cyanoacrylate monomer is 2-octyl cyanoacrylate, abbreviated 2-OCA. In the inventive formulations, the cyanoacrylate monomer can be present in the formulation from about 50% to about 100% by weight, for example from about 75% to about 99%, from about 80% to about 96%, from about 85% to about 94% by weight.

The formulations disclosed herein include one or more cinnamon derivatives dissolved in one or more cyanoacrylate monomers. Cinnamon derivatives useful in the formulations include cinnamaldehyde, cinnamic esters, cinnamon essential oils, and combinations thereof. The cinnamon derivative can be present in any inventive formulation in a concentration from about 0% to about 35% by weight, for example from about 0% to about 15% by weight, from about 3% to about 15%, from about 3% to about 6%, or from about 6% to about 15%. Optionally, the cinnamon derivative can be present in a concentration of at least 3%, at least 4%, at least 5%, or at least 6% by weight. Optionally, the concentration of the cinnamon derivative is up to 10%, up to 20%, up to 25%, up to 30%, or up to 35%.

In some formulations described herein, a cinnamon derivative serves dual functions of providing antimicrobial activity and also functioning as a plasticizer. This dual function is achieved when the cinnamon derivative is present at a weight percent of at least 3%. Accordingly, as one non-limiting example, the dual function of antimicrobial activity and plasticizing can be achieved by using at least 3% by weight of cinnamaldehyde, a cinnamic ester, or a combination thereof. Alternatively, the concentration of the cinnamaldehyde, cinnamic ester, or combination thereof can be at least 4%, at least 5%, or at least 6% by weight. Optionally, the concentration of the cinnamaldehyde, cinnamic ester, or combination thereof can be up to 10%, up to 20%, up to 25%, up to 30%, up to 35%, or up to 40% by weight.

Polymerized films produced from formulations that include the specified concentration of cinnamon derivative have yield strengths similar to polymerized films that include standard plasticizers, such as dibutyl sebacate (DBS). The yield strengths for identical films without plasticizer or the cinnamon derivative are twice as high as for the films with a standard plasticizer or a cinnamon derivative, which indicates a higher elastic modulus and higher stiffness in those materials. Thus, in the specified concentrations, the plasticizing effect of the cinnamon derivatives is an additional benefit beyond antimicrobial activity.

Accordingly, in some examples, a formulation described herein includes a cinnamon derivative selected from cinnamaldehyde, cinnamic esters, and combinations thereof dissolved in a cyanoacrylate monomer, where the cinnamon derivative has a concentration of at least 3%, at least 4%, at least 5%, or at least 6% by weight. Optionally, the concentration of the cinnamon derivative is up to 10%, up to 20%, up to 25%, up to 30%, or up to 35%. Optionally, the formulation is devoid of any plasticizer other than the cinnamon derivative. For example, in some embodiments, the formulation is devoid of DBS. Alternatively, the formulation can include a lower weight percent of the cinnamon derivative and a standard plasticizer, such as but not limited to DBS.

In some embodiments, the formulations described herein include both a cinnamon derivative and triclosan or a derivative thereof. It has been determined that the combination of cinnamaldehyde and triclosan provides a synergistic antimicrobial effect that is greater than the additive antimicrobial effects of the cinnamaldehyde and triclosan individually. For example, combining triclosan with cinnamaldehyde doubles the antimicrobial effect of the cinnamaldehyde on E. coli, even though triclosan alone is known to have minimal impact on E. coli and other gram-negative bacteria. Accordingly, in some examples, a formulation described herein includes a combination of cinnamaldehyde and triclosan or a derivative thereof, dissolved in a cyanoacrylate monomer. Additionally or alternatively, the formulation can include an antimicrobial phenolic derivative other than triclosan.

In any inventive formulation, triclosan or a derivative thereof can be present in concentrations of from about 0% to about 2% by weight, for example from about 100 ppm to about 10,000 ppm, or from about 500 ppm to about 5000 ppm, or from about 1000 ppm to about 5000 ppm. In various examples, the triclosan or derivative thereof can be present in a concentration of up to 750 ppm, up to 1200 ppm, up to 2000 ppm, up to 3500 ppm, up to 5000 ppm, or up to 10,000 ppm. Optionally, the triclosan or derivative thereof can be present in a concentration of at least 500 ppm, at least 750 ppm, at least 1200 ppm, at least 2000 ppm, or at least 3500 ppm. In the inventive formulations that include an antimicrobial phenol derivative, such as triclosan, cinnamaldehyde also can be present in the formulation in concentrations of from about 0% to about 15% by weight, for example, from about 3% to about 15%, from about 3% to about 6%, at least 3%, at least 4%, at least 5%, up to 6%, up to 8%, or up to 10% by weight.

In some embodiments, the formulations described herein can further include povidone-iodine (PVP-I₂) dissolved in the cyanoacrylate monomer. Unlike previously known mixtures of iodine compounds and cyanoacrylates, the povidone-iodine in the inventive formulations is completely dissolved in the cyanoacrylate monomer and is compatible with the monomer. The inventive formulations that include PVP-I₂ optionally also include polyethylene glycol (PEG), which can facilitate solubility and compatibility of the PVP-I₂ in the cyanoacrylate. In any inventive formulation. PVP-I₂ can be present in concentrations of from about 0% to about 3% by weight, such as from about 0.5% to about 3%, from about 0.5% to about 2%, from about 0.5% to about 1% by weight. For example from about 100 ppm to about 700 ppm. The PEG can be present at concentrations from about 0% to about 10% by weight, for example from about 0.5% to about 2% or from about 1% to about 5% by weight. In some examples, an inventive formulation includes PVP-I₂ and PEG in a weight ratio of about 1:10 (PVP-I₂: PEG).

In various examples and unless otherwise stated, any of the formulations described herein optionally further includes a thickener, such as but not limited to a poly (alkyl methacrylate), a poly (alkyl acrylate), a poly(cyanoacrylate), a poly(oxalate), a lactic-glycolic copolymer, a polycaprolactone, a lactic acid-caprolactone copolymer, a polyvinyl acetate, a copolymer of polyacrylate or methacrylate and butadiene, or a polyorthoester. In some examples, the thickener is poly (2-ethylhexyl methacrylate), poly (2-ethylhexyl acrylate), or poly(2-octyl cyanoacrylate). Alternatively, any formulation described herein need not include a thickener. When a thickener is included, the thickener is present from about 0) % to about 20% by weight, for example from about 0% to about 10% by weight.

In various examples and unless otherwise stated, any of the formulations described herein optionally further includes one or more additional agents that is a thickener, a plasticizer, or a polymerization inhibitor. In other examples, any formulation described herein need not include an additional agent that is a thickener, an additional agent that is a plasticizer, or an additional agent that is a polymerization inhibitor. When an additional agent that is a thickener is included, the thickener is present from about 0% to about 20% by weight, for example from about 0% to about 10% by weight. When an additional agent that is a plasticizer is included, the plasticizer is present from about 0% to about 25% by weight, for example from about 0% to about 10% by weight. When an additional agent that is a polymerization inhibitor is present, the polymerization inhibitor is present from about 0 to about 5000 ppm, for example from about 500 to about 1500 ppm.

The effective addition of antimicrobial agents to cyanoacrylate monomer is described herein. The inventive formulations are stable and the cyanoacrylate monomer exhibits a similar cure speed as a control formulation without the antimicrobial agent, so the antimicrobial agents described herein do not adversely affect the film-forming rate of the cyanoacrylate monomer.

### EXAMPLES

The following examples are set forth to illustrate formulations and methods according to some embodiments disclosed herein. These examples are not intended to be inclusive of all aspects of the formulations and methods, but rather to illustrate representative formulations, methods, and results. These examples are not intended to exclude equivalents and variations of the subject matter described herein which are apparent to one skilled in the art.

TABLE 1 lists certain materials used in the example formulations discussed below.

TABLE 1
Materials

| | | |
|---|---|---|
| | | Nominal Amount in |
| | | Formulation (unless |
| | | indicated otherwise) |
| Abbreviation | Name | (g) |

Included in all example formulations identified below

| | | |
|---|---|---|
| 2-OCA | octyl cyanoacrylate | 84 |
| BHA | butylated hydroxy anisole | 0.1 |
| PEHMA | poly (2-ethyl hexyl methacrylate) | 10 |
| SO₂ | sulfur dioxide | 0.012 |
| D&C Violet # 2 | dye | 0.0025 |

Included in some example formulations where indicated

| | | |
|---|---|---|
| PVP-I₂ | povidone iodine | see tables below |
| PEG | polyethylene glycol | see tables below |
| Triclosan | 5-chloro-2-(2,4-dichlorophenoxy)phenol | see tables below |
| CinAl | cinnamaldehyde | see tables below |
| CinEO | cinnamon essential oil | see tables below |
| IsoCin | Isobutyl Cinnamate | see tables below |
| MeCin | Methyl Cinnamate | see tables below |
| DBS | dibutyl sebacate | see tables below |

Some formulations utilize a stock solution of PVP-I₂ in PEG made by adding 0.5 g of PVP-I₂ to 10 g of PEG, and stirring overnight until homogenous.

The compositions used in the Examples and shown in the tables below were made using the following general procedure: BHA was added to 2-OCA monomer with stirring. For formulations including DBS, the DBS was also added to the 2-OCA monomer, and the mixture was stirred for 5 minutes. For formulations including cinnamon derivatives and/or triclosan, the CinAl, CinEO, IsoCin, MeCin, and/or Triclosan were added to the mixture, and the mixture was stirred for 5 minutes. For formulations including PVP-I₂ and PEG, a portion of the PVP-I₂/PEG solution was added and the mixture was stirred for 10 minutes. PEHMA was added slowly into the vortex at ambient temperature. The formulation was further stabilized by presence of SO₂ in the 2-OCA monomer. To the homogenous solution, D&C violet #2 was added. Each resulting cyanoacrylate formulation was used to fill an aluminum tube and sterilized under a dry heat, at a temperature of 125° C. for 60 minutes.

Amounts of certain materials of the example formulations are shown in TABLE 2 and/or in other tables related to specific examples.

TABLE 2
Amounts of certain materials used in example formulations

| | CinAl | CinEO | Triclosan | PVP-I₂ | PEG | DBS |
|---|---|---|---|---|---|---|
| Formulation | (%) | (%) | (ppm) | (ppm) | (%) | (%) |
| Control-1 | 0 | 0 | 0 | 0 | 0 | 6 |
| Control-2 | 0 | 0 | 0 | 0 | 0 | 0 |
| 1 | 5.72 | 5.21 | 0 | 0 | 0 | 0 |
| 2 | 0 | 0 | 2500 | 0 | 0 | 6 |
| 3 | 6 | 0 | 2500 | 0 | 0 | 0 |
| 4 | 0 | 6 | 2500 | 0 | 0 | 0 |
| 5 | 3 | 3 | 2500 | 0 | 0 | 0 |
| 6 | 3 | 3 | 2500 | 0 | 0 | 0 |
| 7 | 6 | 6 | 2500 | 0 | 0 | 0 |
| 8 | 6 | 0 | 0 | 0 | 0 | 0 |
| 9 | 0 | 6 | 0 | 0 | 0 | 0 |
| 10 | 10 | 0 | 2500 | 0 | 0 | 0 |
| 11 | 10 | 0 | 0 | 0 | 0 | 0 |
| 12 | 0 | 0 | 0 | 599 | 1.2 | 6 |
| 13 | 0 | 0 | 1222 | 615 | 1.22 | 6 |
| 14 | 0 | 0 | 2592 | 645 | 1.3 | 6 |

### Example 1: Antimicrobial Properties of Certain Inventive Formulations

The Kirby-Bauer Standard Antimicrobial Susceptibility (ZOI) Test was used to test antimicrobial properties of certain example formulations. In each case, a cured polymer film was prepared by applying the formulation (about 600 mg) on an alcohol cleaned aluminum foil (about 2 in²). After fully curing, the polymer film was carefully removed from the aluminum foil. The film was cut into a 20 mm circle, laid down on a bacteria strain inoculated agar plate, and incubated for 18-24 hours at 37±2° C., according to the ASM guidance as described in Bauer A W, Kirby W M, Sherris J C, Turck M. “Antibiotic susceptibility testing by a standardized single disk method.” Am. J. Clin. Pathol. 45(4):493-6 (1966). Following incubation, the zone sizes were measured to the nearest millimeter using a ruler or caliper. TABLE 3 shows results.

TABLE 3
Summary of inhibition zone size measurements

| | PVP-I₂ | Triclosan | CinAl | CinEO | P. | S. | E. |
|---|---|---|---|---|---|---|---|
| Formulation | (ppm) | (ppm) | (%) | (%) | aeruginosa | aureus | coli |
| Control-1 | 0 | 0 | 0 | 0 | NZ/NI | NZ/I | NZ/I |
| 1 | 0 | 0 | 5.72 | 5.21 | NZ/I | 4 mm | 4 mm |
| *2 | 0 | 2500 | 0 | 0 | NI (NZ) | 1 mm | NI |
| | | | | | | | (NZ) |
| *3 | 0 | 2500 | 6 | 0 | NZ/I | 7 mm | 6 mm |
| 4 | 0 | 2500 | 0 | 6 | NZ/I | 8 mm | NZ/I |
| 5 | 0 | 2500 | 3 | 3 | NZ/I | 6 mm | 3 mm |
| 6 | 0 | 2500 | 3 | 3 | NZ/I | 9 mm | 3 mm |
| 7 | 0 | 2500 | 6 | 6 | NZ/I | 7 mm | 5 mm |
| *8 | 0 | 0 | 6 | 0 | NZ/I | 6 mm | 3 mm |
| *9 | 0 | 0 | 0 | 6 | NZ/I | NZ/I | NZ/I |
| *10 | 0 | 2500 | 10 | 0 | NZ/I | 11 mm | 6 mm |
| *14 | 645 | 2592 | 0 | 0 | NZ/I | 9 mm | NZ/I |

*Lots displaying a synergistic effect

As demonstrated by comparing antibiotic activity of various formulations, certain formulations achieve a synergistic antimicrobial effect of two or more antimicrobial agents.

### Example 2: Tensile Strength of Certain Example Formulations

Tensile Strength Measurements procedure: Tensile strength measures the maximum stress that a material can withstand when stretched or pulled before the sample breaks. Each formulation (2 g) was vortexed for a few seconds with 0.025-0.03 g of 18-Crown-6 ether solution (2.2 w/w %) in isopropanol and poured into a mold to prepare the dog-bone. After fully curing for 24 hours, tensile strength was measured on Instron using a 500 N load cell. The 1^{st} deformation (elastic) force and distance (elongation) were measured to compare flexibilities of cured dog-bones. Results are shown in TABLE 4.

TABLE 4
Summary of tensile strength testing

| | | | | | | 1st peak | 1^{st} peak |
|---|---|---|---|---|---|---|---|
| | DBS | Triclosan | CinAL | IsoCin | MeCin | force | distance |
| Formulation | (%) | (ppm) | (%) | (%) | (%) | (N) | (mm) |
| Control-3 | 0 | 0 | 0 | 0 | 0 | 22.0 ± 3.2 | 6.5 ± 0.6 |
| Control-4 | 6 | 0 | 0 | 0 | 0 | 10.7 ± 2.2 | 7.0 ± 0.5 |
| 15 | 6 | 2500 | 0 | 0 | 0 | 8.9 ± 0.8 | 7.4 ± 0.6 |
| 16 | 0 | 2500 | 6 | 0 | 0 | 9.3 ± 1.4 | 7.8 ± 0.7 |
| 17 | 0 | 2500 | 0 | 6 | 0 | 8.8 ± 0.9 | 7.6 ± 0.9 |
| 18 | 0 | 2500 | 3 | 3 | 0 | 9.6 ± 1.6 | 7.5 ± 0.5 |
| 19 | 0 | 2500 | 5 | 5 | 0 | 8.0 ± 1.4 | 6.8 ± 1.0 |
| 20 | 0 | 0 | 0 | 0 | 6 | 18.5 ± 1.3 | 6.3 ± 0.5 |
| 21 | 0 | 2500 | 0 | 0 | 6 | 16.8 ± 2.2 | 6.4 ± 0.3 |
| 22 | 0 | 2500 | 3 | 0 | 3 | 18.0 ± 2.2 | 6.8 ± 0.5 |
| 23 | 0 | 2500 | 5 | 0 | 5 | 4.4 ± 0.55 | 9.4 ± 1.4 |

### Example 3: Lap Shear Strength of Certain Example Formulations

Lap Shear Strength Measurements procedure: Lap shear strength test uses two ABS sheets (substrates) which are bonded together using the adhesive. Force is applied on the substrate in opposite directions until the substrate separates indicating the bond strength of the adhesive. ABS sheets were cleaned with IPA/Kim wipes and allowed to dry. Adhesive (15-35 mg) is applied evenly to 1×2.5 cm² bond overlap. The excess adhesive is cleaned using Kim wipes and the ABS sheets are clamped with binding clips to apply the same force while curing. These cured samples are tested for lap shear strength using 500 N load cell on Instron. Results are shown in TABLE 5.

TABLE 5
Summary of Lap Shear strength testing
Lap Shear Strength (MPa)

| Formulation | Control-2 | Control-4 | 8 | 3 |
|---|---|---|---|---|
| Average | 2.00 | 1.44 | 1.05 | 1.94 |
| Std. dev. | 0.10 | 0.43 | 0.25 | 0.16 |
| % RSD | 5 | 30 | 24 | 8 |
| Min | 1.73 | 0.71 | 0.63 | 1.65 |
| Max | 2.09 | 2.05 | 1.42 | 2.09 |
| # of data pts | 20 | 19 | 13 | 10 |

### Example 4: Aging Studies of Certain Example Formulations

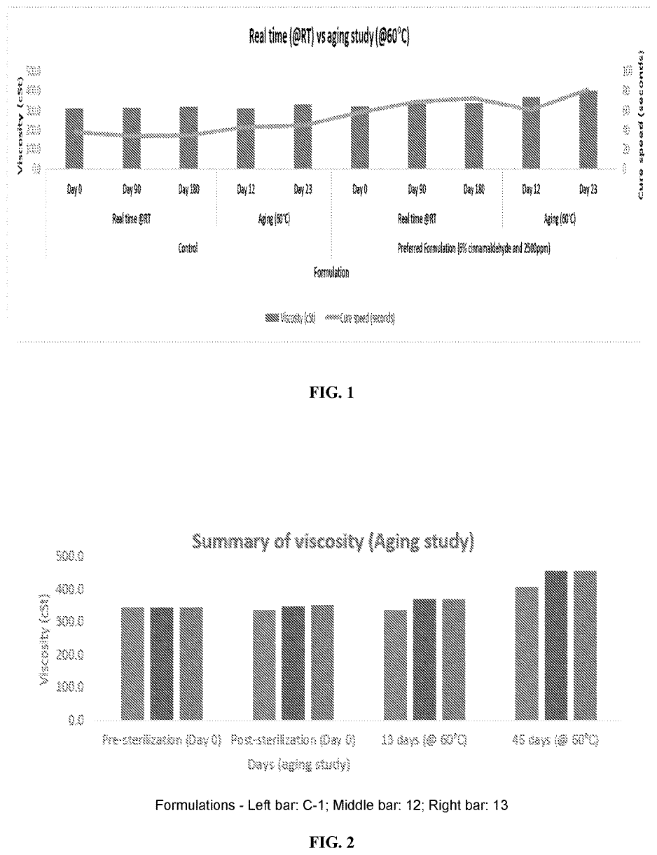

Real time vs. accelerated aging: An aging study comparing real time (at RT) vs. accelerated (at 60° C.) aging was done for control formulation Control-4 and example formulation 24. For real time aging, the formulations were held at room temperature and tested for viscosity and cure speed initially, after about 3 months, and after about six months. For accelerated aging, the formulations were held at 60° C. and tested for viscosity and cure speed at days 12 and 23. Aging stability was better in real time than the corresponding accelerated aging predicted by Arrhenius equation. The real time study at RT for 180 days for viscosity and cure speed showed consistent formulation stability when compared to Day 0 results for cinnamaldehyde formulations. The formulations are shown in TABLE 6, and results are shown in TABLE 7 and FIG. 1.

TABLE 6
Summary of formulations used in real
time vs. accelerated aging study

| Formulation | DBS (%) | CinAl (%) | Triclosan (ppm) |
|---|---|---|---|
| Control-5 | 6 | | 0 |
| 24 | | 6 | 2500 |

TABLE 7
Aging comparison results

| | Control-4 | | | | | 24 | | | | |
|---|---|---|---|---|---|---|---|---|---|---|
| | Real | | | Aging | | Real | | | Aging | |
| | Time @RT | | | (60° C.) | | Time @RT | | | (60° C.) | |
| | Day | Day | Day | Day | Day | Day | Day | Day | Day | Day |
| | 0 | 90 | 180 | 12 | 23 | 0 | 90 | 180 | 12 | 23 |
| Viscosity | 308.4 | 312.9 | 317.1 | 311.2 | 331.4 | 321.7 | 332.6 | 340.6 | 369.8 | 402.6 |
| (cSt) | | | | | | | | | | |
| Cure time | 38.2 | 34.1 | 35 | 43 | 45 | 58 | 69 | 72.2 | 61 | 81.2 |
| (seconds) | | | | | | | | | | |

Accelerated Aging Study: Formulations were filled in aluminum tubes, heat sterilized and packed. These samples were placed in an oven maintained at 60° C. for the aging/stability study. Results are shown in FIG. 2 and TABLE 8.

TABLE 8
Summary of Viscosity results (aging study)

| | | | | Viscosity (cSt) | | | |
|---|---|---|---|---|---|---|---|
| | PVP-I₂ | Triclosan | PEG | Day 0 (Pre- | Day 0 (Post- | Day 13 | Day 46 |
| Formulation | (ppm) | (ppm) | (%) | sterilization) | sterilization) | (60° C.) | (60° C.) |
| Control-1 | 0 | 0 | 0 | 344.7 | 339.6 | 339.6 | 409.5 |
| 12 | 599 | 0 | 1.2 | 346.2 | 348.3 | 371.1 | 459.2 |
| 13 | 615 | 1222 | 1.22 | 346.3 | 354.7 | 373.1 | 456.8 |

### Example 5: Cure Speed of Certain Example Formulations

Formulations were filled in aluminum tubes. The cure time for about 30-50 mg of adhesive was measured at ambient temperature. Results are shown in TABLE 9 and FIG. 3. The weight of each adhesive tube with applicator was measured before and after the application.

TABLE 9
Summary of Cure time results

| | PVP-I₂ | Triclosan | | |
|---|---|---|---|---|
| Formulation | (ppm) | (ppm) | PEG (%) | Cure time (Seconds) |
| Control-1 | 0 | 0 | 0 | 57.4 |
| 12 | 599 | 0 | 1.2 | 87.6 |
| 13 | 615 | 1222 | 1.22 | 66.4 |

### Example 6: Degradation Studies for Cinnamaldehyde Formulations

To check for the leaching of cinnamaldehyde in the formulations the following procedure was followed.

Calibration curve procedure: Cinnamaldehyde stock solution was made in phosphate buffer solution (PBS at 100 mM concentration with pH 7.4). Working standards were prepared at different concentrations using the cinnamaldehyde stock solution. After measuring the absorbance at 300 nM using a UV spectrometer, a calibration curve was established between concentration and absorbance.

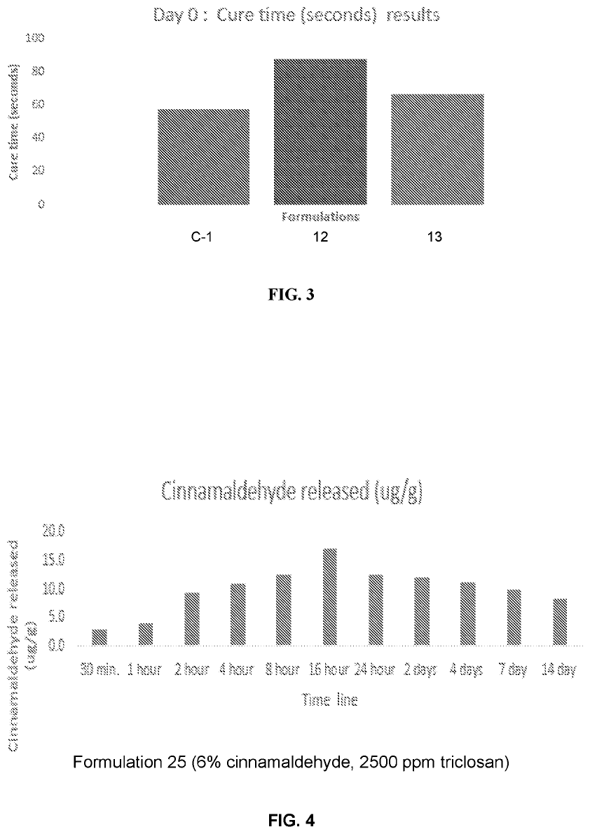

General procedure for measuring the extracted samples: Using formulation 25 with 6% cinnamaldehyde and 2500 ppm triclosan, uniform thin-layer samples (4×6 cm²) were prepared on a PET (polyethylene terephthalate) sheet. The weight of glue used in each sample was nominally 450 mg. After curing these samples for 24 hours at room temperature to form films, each film was placed in a centrifuge tube, and 25 mL of PBS buffer was added. The tubes were sealed with parafilm and placed in a water shaker at 37° C. for different time intervals. After each duration of time indicated in TABLE 10, five tubes were taken out, and the PBS solution was tested for cinnamaldehyde concentration using UV. The concentration of leeched cinnamaldehyde in the PBS solution was determined using the established calibration curve. Five replicates were prepared for every time interval and the average is reported. Results are shown in TABLE 10 and FIG. 4.

TABLE 10
Cinnamaldehyde leaching from film with 6% CinAl and 2500 ppm triclosan

| | Time | | | | | | | | | | |
|---|---|---|---|---|---|---|---|---|---|---|---|
| | 0.5 hr | 1 hr | 2 hr | 4 hr | 8 hr | 16 hr | 24 hr | 2 days | 4 days | 7 days | 14 days |
| CinAl | 2.9 | 3.9 | 9.3 | 11.0 | 12.6 | 17.2 | 12.6 | 12.1 | 11.2 | 10.0 | 8.2 |
| (μg/g) | | | | | | | | | | | |

1. A topical adhesive solution comprising at least one antimicrobial compound dissolved in a cyanoacrylate monomer, wherein the at least one antimicrobial compound comprises a cinnamon derivative selected from cinnamaldehyde, a cinnamic ester, or a cinnamon essential oil.

2. The topical adhesive solution of claim 1, wherein the cinnamon derivative is present in a range from about 3% to about 35% by weight.

3. The topical adhesive solution of claim 2, wherein the cinnamon derivative is present in a range from about 3% to about 10% by weight.

4. The topical adhesive solution of claim 1, wherein the cinnamon derivative comprises cinnamaldehyde or a cinnamon essential oil, and wherein the at least one antimicrobial compound further comprises triclosan or a derivative thereof.

5. The topical adhesive solution of claim 4, wherein the cinnamon derivative is present in a range from about 2% to about 30% by weight.

6. The topical adhesive solution of claim 5, wherein the cinnamon derivative is present in a range from about 3% to about 10% by weight.

7. The topical adhesive solution of claim 4, wherein the triclosan or derivative thereof is present in a range from about 100 ppm to about 10,000 ppm.

8. The topical adhesive solution of claim 7, wherein the triclosan or derivative thereof is present in a range from about 1000 ppm to about 5000 ppm.

9. The topical adhesive solution of claim 1, wherein the at least one antimicrobial compound further comprises povidone iodine.

10. The topical adhesive solution of claim 9, wherein the povidone iodine is present in a range from about 0.5% to about 3% by weight.

11. The topical adhesive solution of claim 10, wherein the povidone iodine is present in a range from about 0.5% to about 2% by weight.

12. The topical adhesive solution of claim 10, wherein the triclosan or derivative thereof is present in a range from about 100 ppm to about 10000 ppm.

13. The topical adhesive solution of claim 12, wherein the triclosan or derivative thereof is present in a range from about 1000 ppm to about 5000 ppm.

14. The topical adhesive solution of claim 1, wherein the solution further comprises polyethylene glycol.

15. The topical adhesive solution of claim 15, wherein the polyethylene glycol is present in a range from about 1% to about 10% by weight.

16. The topical adhesive solution of claim 16, wherein the polyethylene glycol is present in a range from about 1% to about 5% by weight.

17. The topical adhesive solution of claim 1, further comprising a thickener.

18. The topical adhesive solution of claim 18, wherein the thickener comprises poly(2-ethylhexyl methacrylate), poly(2-ethylhexyl acrylate), or poly(2-octyl cyanoacrylate).

19. The topical adhesive solution of claim 1, further comprising a polymerization accelerator.

20. The topical adhesive solution of claim 20, wherein the polymerization accelerator comprises a crown ether or a calixarene.

21. The topical adhesive solution of claim 1, wherein the formulation does not comprise a plasticizer.

22. The topical adhesive solution of claim 1, wherein the formulation further comprises a plasticizer

23. The topical adhesive solution of claim 23, wherein the plasticizer comprises dibutyl sebacate, tributyl citrate, or tributyl O-acetyl citrate.

24. The topical adhesive solution of claim 1, wherein the formulation further comprises a polymerization inhibitor.

25. The topical adhesive solution of claim 25, wherein the polymerization inhibitor comprises butylated hydroxy anisole or sulfur dioxide.

26. The topical adhesive solution of claim 1, wherein the formulation is homogeneous.

27. The topical adhesive solution of claim 1, wherein the formulation is stable.
